(19) United States
(12) Patent Application Publication
(10) Pub. No.: US 2004/0247686 A1
(43) Pub. Date: Dec. 9, 2004

(54) Pharmaceutical compositions comprising epinastine for the treatment of skin diseases

(75) Inventors: Minoru Okada, Inzai (JP); Akira Takahashi, Kitasouma-gun (JP); Norimitsu Umehara, Tokorozawa-shi (JP); Tetsuo Hayashi, Tomisato-shi (JP); Yoichi Onuki, Narita-shi (JP); Kazuki Matsumoto, Sakura (JP)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/803,674

(22) Filed: Mar. 18, 2004

(30) Foreign Application Priority Data
Apr. 4, 2003 (EP) EP 03 007 779
Apr. 4, 2003 (EP) EP 03 007 759
Apr. 9, 2003 (EP) EP 03 007 778
Apr. 17, 2003 (EP) EP 03 008 989

(51) Int. Cl. A61K 9/14; A61K 38/00; A61K 31/137

(52) U.S. Cl. 424/488; 514/649; 514/2

(57) ABSTRACT

The present invention relates to new pharmaceutical compositions for the treatment of skin disease. The composition comprises an antihistaminic-effective amount of Epinastine or a pharmaceutically acceptable salt thereof as a pharmacologically active compound and at least one compound selected from the group consisting of one or more sulfur containing amino acid(s) or peptide(s) as biologically active donor of a —S— or —SH group, at least one vitamin of the B group, at least one vitamin having antioxidant properties and an antiphlogistic-effective amount of an antiphlogistic compound. The compositions also may comprise pharmaceutically acceptable additives.

### TECHNICAL FIELD

The present invention relates to new pharmaceutical compositions for the treatment of skin diseases. The compositions comprise an antihistaminic-effective amount of Epinastine or a pharmaceutically acceptable salt thereof as a pharmacologically active compound and at least one further compound selected from the group consisting of a) sulfur containing amino acid(s) or peptide(s) as biologically active donor of a —S— or —SH group, b) vitamins of the B group, c) vitamins having antioxidant properties and d) antiphlogistic compounds. The compositions also may comprise pharmaceutically acceptable additives, carriers and excipients.

The invention also relates to the use of these formulations for the treatment of pruritus (itching) derived from skin disease such as urticaria, eczema, and skin irritation.

Remarkably, the compositions described in the present invention are highly effective in the treatment of skin diseases associated with allergic reactions.

### BACKGROUND OF THE INVENTION

In recent years, the incidence of developing skin diseases associated with allergic reactions has increased due to changes in diet, changes of the life style, air pollution, increased exposure to environmental chemicals, from numerous environmental deterioration, stress in the social life and so on. Among these allergic reactions are urticaria, eczema, skin irritation, and dermatitis as well as skin diseases accompanying itching represented by pruritus, prurigo, psoriasis vulgaris, etc.

Urticaria, a synonym of wheal, is a transient edema. The disease is characterized by a sudden onset of itchy sensation on skin, followed by developing well defined eruption swelling up like weal and growing into a size of nail plate to palm exacerbated by scratching. Although the symptoms disappear within a couple of minutes to hours and may not leave any skin disorder, episodes of development into eruption are likely to recur. Causes of urticaria may include autosensitization, sensitizations associated with difficult menstruation, pregnancy, foods, medicines and insect stings, abnormal responses to heat, cold, mechanical stimuli and light, remote responses to bacterial infections, gastrointestinal, hepatic, and renal disease, an endocrinopathic involvement, and psychological factors.

Eczema or dermatitis is the most major skin disease, characterized by inflammatory response on skin. Eczema and dermatitis are often referred altogether as eczematous dermatitis group. The diseases are often caused by pathological interactions caused by external stimuli (numbers of chemicals, fragrances, metals, detergents, medicines, plants, bacteria, insects, sunlight, heat, cold, dryness), internal abnormalities (local abnormalities such as perspiration, abnormal sebum secretion, abnormal keratosis, and systemic abnormalities such as atopic disposition, infection site, digestive disorder, renal dysfunction, endocrine disturbance), and bodily condition. Eczematous dermatitis group includes contact dermatitis, atopic dermatitis, seborrheic dermatitis, nummular eczema, autosensitization dermatitis, and lichen simplex chronicus Vidal.

Housewives' eczema, keratodermia tylodes palmaris progressiva, diaper dermatitis, and photocontact dermatitis are classified as atypical contact dermatitis. In addition, the group may include diffuse neurodermatitis, stasis dermatitis, infectious eczematoid dermatitis, and perioral dermatitis. Broadly, it may also include radiodermatitis, scald (burn), and frostbite.

Pruritus is a disease characterized by an onset of itchy sensation (itching) on apparently normal skin. Range of affected lesion divides pruritus into universal pruritus and localized pruritus. The disease is derived from a variety of causes, and often develops as a symptom of systemic disease.

Prurigo presents extreme itching and is papule or urticaria-like nodule that progress to chronic or recurrent disorder, and can be broadly classified into prurigo acuta including strophulus infantum, lichen urticatus, prurigo aestiralis, prurigo simplex acuta, prurigo subacuta such as prurigo simplex subacuta, and prurigo chronica including chronica multiformis, prurigo nodularis, prurigo Hebra, and prurigo simplex chronica. Mechanisms of the pathogenesis are unrevealed. Insect sting in prurigo acuta, and diabetes mellitus, hepatopathy, leukemia, Hodgkin's disease, visceral cancer, and polycythemia in prurigo chronica are thought of as causatives.

Psoriasis vulgaris is an inflammatory skin disease, and presents histological characteristics of epidermal hyperplasia and inflammatory cellular infiltration. Eruption typically develops on head, extension side of extremities, and some parts of truncus which are in particular likely to come in contact with mechanical compression, in almost a half of which pruritus is observed. Immunological abnormalities may be concerned as a cause of disease.

It is emphasized that improvements on surroundings such as eliminating causative antigens is the most important treatment of these skin diseases, particularly for allergic skin disease. Nevertheless, as already reviewed, pathogenic causes are complicated, and therefore are fallible to be identified. Consequently, compositions combining antihistaminic compounds are of the frequent choice for treatments of these symptoms including itchy sensation caused from skin diseases.

Compositions comprising Epinastine in combination with vitamins of the B group have already been described.

Liquid-type formulations for cold and rhinitis combined with loxoprofen sodium, dihydrocodeine phosphate, Epinastine hydrochloride, dl-methylephedrine hydrochloride, ambroxol hydrochloride, anhydrous caffeine, vitamin B₁ nitrate, and vitamin B₂ as pharmacological active compounds are disclosed in Example 4 of Publication of Japanese Patent Application JP2001-199882A.

Liquid-type formulations for cold combined with loxoprofen sodium, dihydrocodeine phosphate, Epinastine hydrochloride, dl-methylephedrine hydrochloride, ambroxol hydrochloride, anhydrous caffeine, vitamin B₁ nitrate, and vitamin B₂ as pharmacological active compounds are disclosed in Example 4 of Publication of Japanese Patent Application JP2001-172175A.

Tablet-type antitussive agent for cold combined with ibuprofen, Epinastine hydrochloride, noscapine, benproperine phosphate, ambroxol hydrochloride, trimetoquinol hydrochloride, anhydrous caffeine, vitamin B₁ nitrate, and vitamin B₂ as pharmacological active compounds is disclosed in Example 4 of Publication of Japanese Patent Application JP10-017473A.

The aforementioned examples are combination medicines of Epinastine, vitamin B₁, vitamin B₂, etc. All these medicines are cold remedies.

Therefore, the use of a combination of Epinastine and a vitamin of the B group in the treatment of skin diseases in association with allergic reactions is new.

Compositions comprising Epinastine in combination with vitamins having antioxidant properties have also already been described.

A liquid antitussive formulation composed of acetaminophen, dimemorfan phosphate, Epinastine hydrochloride, dl-methylephedrine hydrochloride, bromhexine hydrochloride, lysozyme chloride, anhydrous caffeine, and vitamin C as pharmacological active compounds is disclosed in Example 5 of JP2001-097856A.

A liquid antitussive formulation composed of naproxen, dihydrocodeine phosphate, Epinastine hydrochloride, dl-methylephedrine hydrochloride, bromhexine hydrochloride, lysozyme chloride, anhydrous caffeine, and vitamin C as pharmacological active compounds is disclosed in Example 29 of JP2000-344682A.

A liquid medical composition having antitussive effect composed of fenoprofen, dihydrocodeine phosphate, Epinastine hydrochloride, dl-methylephedrine hydrochloride, bromhexine hydrochloride, lysozyme chloride, anhydrous caffeine, and vitamin C as pharmacological active compounds is disclosed in Example 5 of JP11-071281A.

A liquid cough medicine comprising fenoprofen, dihydrocodeine phosphate, Epinastine hydrochloride, dl-methylephedrine hydrochloride, ambroxol hydrochloride, anhydrous caffeine, and vitamin C as pharmacological active compounds is disclosed in Example 5 of JP11-071281A.

All these medicines comprising Epinastine and a vitamin with antioxidant properties are antitussive expectorant and cold remedies. The use thereof for the treatment of skin diseases has not been disclosed.

Compositions comprising Epinastine in combination with an antiphlogistic has already been disclosed.

A tablet formulation composed of phenylephrine hydrochloride, Epinastine hydrochloride, isopropamide iodide, glycyrrhizinate dipotassium, lidocaine hydrochloride, and anhydrous caffeine as pharmacological active compounds is disclosed in Example 3 of JP10-298107A.

This example is a combination drug composed of such as Epinastine and glycyrrhizinate dipotassium. The drug has extremely potent suppressive action on airway hypersecretion or cold syndrome therapeutic agent, and is not a treatment for skin disease.

### SUMMARY OF THE INVENTION

The present invention aims to provide compositions for the treatment of skin diseases that exert its significant utility to achieve effective improvements.

In addition, the present invention intends to provide the compositions for treatment of skin diseases by employing highly effective pharmaceutical compounds for significant improvements on symptoms of skin diseases accompanying itching, particularly urticaria, eczema, skin fit, dermatitis, pruritus, eruption, and psoriasis vulgaris accompanying itchy sensation.

### DETAILED DESCRIPTION OF THE INVENTION

The present invention relates to pharmaceutical compositions for the treatment of skin disease, whereas the compositions comprise an antihistaminic-effective amount of Epinastine or a pharmaceutically acceptable salt thereof as pharmacologically active compound and at least one further compound selected from the group consisting of a) sulfur containing amino acid(s) or peptide(s), b) vitamins of the B group, c) vitamins having antioxidant properties and d) antiphlogistic compounds. Preferably, the vitamins having antioxidant properties are free radical scavenger.

Epinastine, (±) 3-amino-9, 13b-dihydro-1H-dibenz [c, f] imidazo [1,5-a] azepine, the hydrochloride thereof respectively, is a drug possessing H1-antihistaminic property. It primarily has been used to treat allergic reaction of the eyes and the nasal mucosa.

In all compositions of the present invention Epinastine preferably is taken in the form of a salt such as the hydrochloride, hydrobromide, oxalate, nitrate, sulfonate, fumarate, maleate, sulfate, and phosphate. The free base can be taken, too. Preferred is Epinastine-hydrochloride.

The amount of Epinastine or a pharmacologically acceptable salt thereof depends on the application route.

In the case of oral application, the daily dosage in equivalent quantity of Epinastine-hydrochloride for an adult is between 2 and 20 mg, preferably between 5 and 15 mg, and further more preferably between 7.5 and 12.5 mg. preferably, this amount is given via one or more dosage units, like tablets.

In the case of topical application the amount in equivalent quantity of Epinastine hydrochloride is between 1 and 50 mg per 1 g of composition, preferably between 2 and 30 mg per 1 g of composition, and further more preferably between 5 and 15 mg per 1 g of composition.

In one embodiment of the invention, the pharmaceutical compositions for the treatment of skin diseases of the present invention comprise Epinastine and sulfur containing amino acid(s) or peptide(s).

The sulfur containing amino acid(s) or peptide(s) shall act as biologically active donor(s) of a —S— or a —SH group. Sulfur containing amino acids are known to maintain or activate enzyme activities and thereby exert a biochemical reaction in which the SH group is involved.

In the context of the present invention the sulfur containing amino acid(s) or peptide(s) can be used as such or in the form of a pharmaceutically acceptable salt or as derivatives thereof.

Examples of these sulfur containing amino acid(s) or peptide(s) comprise cysteine, methionine, aminoethylsulfonic acid (taurine), glutathione, cystine, homocysteine, homocystine, cysteine sulfinic acid, lanthionine, mixtures thereof as well as their pharmaceutically acceptable salts or derivatives. It is also possible to use the mixed disulfides of any of the aforementioned compounds having a thiol-group. However, homogeneous disulfides are preferred among the disulfides. It is preferred to use one or more of these acids, particularly preferred are cysteine, methionine, taurine and glutathione as well as their pharmaceutically acceptable salts or derivatives.

The amount of the sulfur containing amino acid varies in dependency of the type, the combination chosen and the application route.

For oral use the daily dosage for an adult lies in the range of from 5 to 10000 mg, and for topical use it lies in the range of from 0.01 to 200 mg.

L-Cysteine is one of the preferred sulfur containing amino acids to be used in the context of the present invention. For oral use the daily dosage for an adult lies normally in the range of from 5 to 1000 mg, preferably in the range of from 10 to 480 mg, and more preferably in the range of from 20 to 240 mg.

For topical use, the dosage is up to 200 mg per 1 g of composition, preferably between 0.01 and 50 mg per 1 g of composition, and more preferably between 0.1 and 15 mg per 1 g of composition.

L-Methionine is used in oral formulations in daily dosages for an adult of between 0.5 and 5000 mg, preferably between 1 and 3000 mg, and more preferably between 2 and 1000 mg.

For topical use the dosage is up to 200 mg per 1 g of composition, preferably between 0.01 and 50 mg per 1 g of composition, and more preferably between 0.1 and 15 mg per 1 g of composition.

Aminoethylsulfonic acid, known as taurine or 2-aminoethylsulfonic acid, is given in daily dosages for an adult if applied orally which are between 5 and 10000 mg, preferably between 25 and 5000 mg, and more preferably between 30 and 3000 mg.

For topical use the dosage is up to 200 mg per 1 g of composition, preferably between 0.01 and 50 mg per 1 g of composition, and more preferably between 0.1 and 15 mg per 1 g of composition.

Glutathione, γ-L-glutamyl-L-cysteinyl-glycine is given in daily dosages for an adult if applied orally which are between 5 and 1000 mg, preferably between 25 and 600 mg, an more preferably between 50 and 300 mg.

For topical use the dosage is up to 200 mg per 1 g of composition, preferably between 0.01 and 50 mg per 1 g of composition, and more preferably between 0.1 and 15 mg per 1 g of composition.

Dose adjustment of Epinastine and sulfur containing amino acid(s) or peptide(s) may reflect age, body weight, and manifesting symptoms. Epinastine and the sulfur containing amino acid(s) or peptide(s) can be combined together in one pharmaceutical preparation or the two components are formulated separately from each other in two pharmaceutical preparations and then given together or in close timely proximity, i.e. within 12 hours, preferably within 1 hour more preferably within 15 minutes and in particular preferred within 2 minutes. Preferred are pharmaceutical compositions that contain both ingredients, i.e. the both ingredients are not separated.

According to the invention there is also provided a pharmaceutical formulation for the treatment of skin diseases including at least one vitamin of the B group in addition to Epinastine. B group vitamins are regarded as a vitamin group having important influences on metabolism of protein, lipid, and carbohydrate by becoming components of coenzyme in the human vivo, or by being coenzyme itself, and help normalize the organism such as skin, nail, hair, and mucosa.

B group vitamins used in the pharmaceutical formulations for treatment of skin disease described in the present invention include vitamin-like active substances such as vitamin B₁ such as thiamine, thiamine hydrochloride, thiamine nitrate, thiamine disulfide nitrate, thiamine disulfide, thiamine dicetylsulfate salt, dicethiamine hydrochloride, fursultiamine hydrochloride, fursultiamine, octotiamine, cycotiamine, bisibutiamine, bisbentiamine, prosultiamine, benfotiamine, cocarboxylase and dibenzoylthiamaine, and its salt and derivatives thereof, vitamin B₂ such as riboflavin, riboflavin butyrate, riboflavin sodium phosphate and flavin adenine dinucleotide, and its salt and derivatives thereof, vitamin B₆ such as pyridoxine, pyridoxal, pyridoxamine, pyridoxine phosphate, pyridoxal phosphate and pyridoxamine phosphate, and its salt and derivatives thereof, vitamin B₁₂ such as cobalamin, cyanocobalamin, hydroxocobalamin, hydroxocobalamin acetate and mecobalamin, and its salt and derivatives thereof, niacin such as nicotinic acid, nicotinamide, inositol hexanicotinate and hepronicate, and its salt and derivatives thereof, pantothenic acid such as calcium pantothenate, sodium pantothenate, panthenol and pantethine, and its salt and derivatives thereof, biotin, vitamins such as folic acid, orotic acid such as orotic acid and choline orotate, and its salt and derivatives thereof, thioctic acid such as thioctic acid (lipoic acid) and thioctic acid amide, and its salt and derivatives thereof, p-aminobenzoic acid and its salt and derivatives thereof, inositol such as inositol and inositol hexanicotinate, and its salt and derivatives thereof, carnitine such as carnitine, carnitine chloride and acetyl-carnitine and its salt and derivatives thereof, and choline such as choline and choline orotate and its salt and derivatives thereof.

One or more compounds of these B group vitamins can be used to formulate this invention.

Preferred are the following combinations of Epinastine plus one vitamin of the vitamin B group:

Epinastine plus

vitamin B₁,

vitamin B₂,

vitamin B₆,

vitamin B₁₂,

niacin,

pantothenic acid,

biotin,

folic acid,

orotic acid,

thioctic acid,

p-aminobenzoic acid,

inositol,

carnitine,

choline, or a salt or derivatives of each.

If the combination shall comprise at least two vitamins of the vitamin B group, the two vitamins preferably are:

riboflavin or riboflavin butyrate and pyridoxine hydrochloride,

thiamin nitrate and riboflavin or riboflavin butyrate,

pyridoxine hydrochloride and thiamin nitrate,

nicotinamide and pyridoxine hydrochloride,

nicotinamide and thiamin nitrate,

nicotinamide and riboflavin or riboflavin butyrate,

pyridoxine hydrochloride and tocopherol acetate.

If the combination shall comprise at least three vitamins of the vitamin B group, the three vitamins preferably are:

thiamin nitrate, riboflavin or riboflavin butyrate, pyridoxine hydrochloride,

thiamin nitrate, riboflavin or riboflavin butyrate, nicotinamide,

thiamin nitrate, nicotinamide, pyridoxine hydrochloride,

nicotinamide, riboflavin or riboflavin butyrate, pyridoxine hydrochloride,

pyridoxine hydrochloride, riboflavin sodium phosphate, panthenol.

If the combination shall comprise at least four vitamins of the vitamin B group, the four vitamins preferably are:

thiamin nitrate, riboflavin butyrate, pyridoxine hydrochloride, nicotinamide.

Of any of the named B vitamins another salt form may be used instead of the named one.

Furthermore, other pharmaceutical active substances may be combined to formulate this invention in addition to Epinastine and B group vitamins. Examples comprise of sulfur-containing amino acid such as cysteine, methionine, aminoethylsulfonic acid and glutathione, antioxidant vitamins such as vitamin C, vitamin E and vitamin A, antioxidant vitamin-like substances such as ubiquinone, pangamic acid and flavonoid, D group vitamins such as ergocalciferol and cholecalciferol.

Although combination amount of B group vitamins to formulate the present invention varies depending on types of B group vitamins, for oral use given daily to an adult it lies in the range from 0.0001 to 1500 mg, and for topical use it lies in the range from 0.1 to 200 mg/g.

In further details, combination amount of vitamin B₁ and its salt and derivatives thereof for oral use given daily to an adult lies normally in the range from 0.1 to 500 mg, preferably in the range from 0.5 to 200 mg, and more preferably in the range from 1 to 100 mg, and for topical use it lies normally in the range within 200 mg/g, preferably in the range from 0.1 to 50 mg/g, and more preferably in the range from 1 to 15 mg/g.

Combination amount of vitamin B₂ and its salt and derivatives thereof for oral use given daily to an adult lies normally in the range from 0.5 to 180 mg, preferably in the range from 1 to 90 mg, and more preferably in the range from 2 to 45 mg. And for topical use it lies normally in the range within 200 mg/g, preferably in the range from 0.1 to 50 mg/g, and more preferably in the range from 1 to 15 mg/g.

Combination amount of vitamin B₆ and its salt and derivatives thereof for oral use given daily to an adult lies normally in the range from 0.1 to 500 mg, preferably in the range from 1 to 200 mg, and more preferably in the range from 5 to 100 mg. And for topical use it lies normally in the range within 200 mg/g, preferably in the range from 0.1 to 50 mg/g, and more preferably in the range from 1 to 15 mg/g.

Combination amount of vitamin B₁₂ and its salt and derivatives thereof for oral use given daily to an adult lies normally in the range from 0.0001 to 15 mg, preferably in the range from 0.0005 to 3 mg, and more preferably in the range from 0.001 to 1.5 mg. And for topical use it lies normally in the range within 200 mg/g, preferably in the range from 0.1 to 50 mg/g, and more preferably in the range from 1 to 15 mg/g.

Combination amount of niacin and its salt and derivatives thereof for oral use given daily to an adult lies normally in the range from 0.1 to 1000 mg, preferably in the range from 1 to 800 mg, and more preferably in the range from 12 to 400 mg. And for topical use it lies normally in the range within 200 mg/g, preferably in the range from 0.1 to 50 mg/g, and more preferably in the range from 1 to 15 mg/g.

Combination amount of pantothenic acid and its salt and derivatives thereof for oral use given daily to an adult lies normally in the range from 0.1 to 120 mg, preferably in the range from 1 to 60 mg, and more preferably in the range from 5 to 30 mg. And for topical use it lies normally in the range within 200 mg/g, preferably in the range from 0.1 to 50 mg/g, and more preferably in the range from 1 to 15 mg/g.

Combination amount of biotin for oral use given daily to an adult lies normally in the range from 0.001 to 10 mg, preferably in the range from 0.005 to 1 mg, and more preferably in the range from 0.01 to 0.5 mg. And for topical use it lies normally in the range within 200 mg/g, preferably in the range from 0.1 to 50 mg/g, and more preferably in the range from 1 to 15 mg/g.

Combination amount of folic acid for oral use given daily to an adult lies normally in the range from 0.01 to 100 mg, preferably in the range from 0.05 to 20 mg, and more preferably in the range from 0.1 to 10 mg. And for topical use it lies normally in the range within 200 mg/g, preferably in the range from 0.1 to 50 mg/g, and more preferably in the range from 1 to 15 mg/g.

Combination amount of orotic acid and its salt and derivatives thereof for oral use given daily to an adult lies normally in the range from 1 to 500 mg, preferably in the range from 5 to 200 mg, and more preferably in the range from 10 to 100 mg. And for topical use it lies normally in the range within 200 mg/g, preferably in the range from 0.1 to 50 mg/g, and more preferably in the range from 1 to 15 mg/g.

Combination amount of thioctic acid and its salt and derivatives thereof for oral use given daily to an adult lies normally in the range from 0.1 to 500 mg, preferably in the range from 1 to 200 mg, and more preferably in the range from 2 to 100 mg. And for topical use it lies normally in the range within 200 mg/g, preferably in the range from 0.1 to 50 mg/g, and more preferably in the range from 1 to 15 mg/g.

Combination amount of p-aminobenzoic acid and its salt and derivatives thereof for oral use given daily to an adult lies normally in the range from 1 to 1500 mg, preferably in the range from 2 to 1000 mg, and more preferably in the range from 10 to 500 mg. And for topical use it lies normally in the range within 200 mg/g, preferably in the range from 0.1 to 50 mg/g, and more preferably in the range from 1 to 15 mg/g.

Combination amount of inositol and its salt and derivatives thereof for oral use given daily to an adult lies normally in the range from 1 to 800 mg, preferably in the range from 5 to 400 mg, and more preferably in the range from 10 to 200 mg. And for topical use it lies normally in the range within 200 mg/g, preferably in the range from 0.1 to 50 mg/g, and more preferably in the range from 1 to 15 mg/g.

Combination amount of carnitine and its salt and derivatives thereof for oral use given daily to an adult lies normally in the range from 1 to 1000 mg, preferably in the range from 2 to 600 mg, and more preferably in the range from 10 to 100 mg. And for topical use it lies normally in the range within 200 mg/g, preferably in the range from 0.1 to 50 mg/g, and more preferably in the range from 1 to 15 mg/g.

Combination amount of choline and its salt and derivatives thereof for oral use given daily to an adult lies normally in the range from 1 to 1500 mg, preferably in the range from 2 to 1000 mg, and more preferably in the range from 10 to 500 mg. And for topical use it lies normally in the range within 200 mg/g, preferably in the range from 0.1 to 50 mg/g, and more preferably in the range from 1 to 15 mg/g.

It is possible that the pharmaceutical formulations comprising Epinastine and one or more compounds of the vitamin B group are orally given all at once or in divided doses. For topical purposes the daily amount can be applied all at once or it can be divided in doses. The topical application should occur directly onto the affected region of skin. Dose adjustment of Epinastine and B group vitamins may reflect age, body weight, and manifesting symptoms.

In addition, when the pharmaceutical formulations comprising Epinastine and one or more compounds of the vitamin B group are orally given, part of or all of B group vitamins, may be formulated in a slow release form while Epinastine itself or a combination of Epinastine and B group vitamins are formulated for instant release. When other additional active components are present, they may be in either of the two formulation parts, the instant or the slow release part of the formulation in accordance with the pharmacokinetic characteristic of each active component.

Epinastine and the at least one vitamin of the B group can be combined together in one pharmaceutical preparation or the two combinations are formulated separately from each other in two pharmaceutical preparations and then given together or in close timely proximity, i.e. within 12 hours, preferably within 1 hour more preferably within 15 minutes and in particular preferred within 2 minutes. Preferred are pharmaceutical compositions that contain both ingredients, i.e. the both ingredients are not separated.

These fast release components and slow release components may be present in one application unit each. The two components may be formulated separately and then they are combined physically in one dosage unit, f. e. a capsule or the like or they are applied together. In an alternative embodiment the fast release components and slow release components may be regarded as one unit formulation. Such unit formulations may include for example multilayer tablets combining fast release layer(s) and slow release layer(s), granules combining fast release granules and slow release granules or capsules filled with the granules, hard capsules filled with a combination of small fast release tablet(s) and slow release tablet(s), and dry syrup or suspension syrup using microcapsule or microsphere as slow release components.

In a further embodiment of the present invention the pharmaceutical compositions for the treatment of skin diseases comprise at least one antioxidant vitamin in addition to Epinastine.

There is no particular restriction in types of the antioxidant vitamin(s) to be together with Epinastine provided that the corresponding vitamin(s) has (have) antioxidant properties.

In the context of the present invention preferred examples include vitamin C, vitamin E, vitamin A, and such vitamin-like active substances.

Concerning the vitamin C/vitamin C-like active substances, the at least one vitamin having antioxidant properties preferably may be selected from one or more of the following group: ascorbic acid, metallic ascorbate, such as sodium ascorbate, potassium ascorbate, calcium ascorbate, magnesium ascorbate, aluminum ascorbate, ascorbic acid derivative, such as ascorbyl phosphates, in particular sodium or potassium ascorbyl phosphate, magnesium ascorbyl phosphate, calcium ascorbyl phosphate, and aluminum ascorbyl phosphate, ascorbic sulfates such as disodium ascorbyl sulfate, potassium ascorbyl sulfate, magnesium ascorbyl sulfate, calcium ascorbyl sulfate, and aluminum ascorbyl sulfate, ascorbyl glucosides such as ascorbyl-2-glucoside, ascorbyl fatty acid glucosides, ascorbyl fatty acids, erythorbic acid (isoascorbic acid), and metallic erythorbate, such as sodium erythorbate. Examples of vitamin E/vitamin E-like active substances comprise d-α-tocopherol, dl-α-tocopherol, d-α-tocopherol acetate, dl-α-tocopherol acetate, d-α-tocopherol succinate, dl-α-tocopherol succinate, dl-α-tocopherol calcium succinate, tocopherol nicotinate, vitamin E linoleate (preferably a mixture of tocopheryl esters, mainly tocopheryl linoleate), dl-βtocopherol, dl-γtocopherol, d-δ-tocopherol, and natural mixed tocopherol.

Examples of vitamin A/vitamin A-like active substances comprise vitamin A, retinal acetate, retinol palmitate, retinol etretinate, vitamin A oil, cod liver oil, strong cod liver oil, and also carotene such asα-carotene, β-carotene, γ-carotene, and lycopene can be added to the above.

Examples of other vitamin-like active substance which has antioxidant properties comprise of ubiquinone (coenzyme Q, ubidecarenone), pangamic acid, and flavonoids.

One or more compounds of these antioxidant vitamins can be used to formulate the antioxidant vitamin comprising composition of this invention. Preferably the composition contains only one of the named vitamins.

For combinations of Epinastine plus two vitamins the combinations with

vitamin C plus vitamin E,

vitamin C plus vitamin A, and

vitamin C plus such vitamin-like active substances

vitamin E plus vitamin A, and

vitamin E plus such vitamin-like active substances are preferred.

For combinations of Epinastine plus three vitamins the combinations with

vitamin C, vitamin A and vitamin E are preferred.

Furthermore, other pharmaceutical active substances can be combined with Epinastine and the antioxidant vitamins to formulate this invention. Examples comprise sulfur amino acids such as cysteine, methionine, aminoethylsulfonic acid or glutathione. Other examples are vitamin D such as ergocalciferol and cholecalciferol. However, the combination also may comprise any other kind of vitamin or vitamin mixture.

In the formulation comprising Epinastine and the antioxidant vitamin the amount of the at least one antioxidant vitamin varies depending on the type of the antioxidant vitamin. For daily oral use for an adult, it lies in the range of from 0.01 to 3000 mg, and for topical use, it lies in the range of from 0.1 to 200 mg/g.

In particular, the daily dosage range for a vitamin C, given orally to an adult lies in the range of from 5 to 3000 mg, preferably in the range of from 25 to 2000 mg, and more preferably in the range of from 50 to 500 mg, and for topical use it lies in the range within 200 mg/g, preferably in the range of from 0.1 to 50 mg/g, and more preferably in the range of from 5 to 40 mg/g.

The daily dosage range for a vitamin E, given orally to an adult lies in the range of from 1 to 500 mg, preferably in the range of from 5 to 300 mg, and more preferably in the range of from 10 to 100 mg. And for topical use it lies in the range within 200 mg/g, preferably in the range of from 0.1 to 60 mg/g, and more preferably in the range of from 0.5 to 30 mg/g.

The daily dosage range for a vitamin A, given orally to an adult lies in the range of from 10 to 10000 IU (international unit), preferably in the range of from 100 to 4000 IU, and more preferably in the range of from 500 to 2000 IU. And for topical use it lies in the range within 200000 IU/g, preferably in the range of from 100 to 50000 IU/g, and more preferably in the range of from 1000 to 10000 IU/g.

The daily orally to an adult given dosage range for ubiquinone, (coenzyme Q, ubidecarenone), which is vitamin-like active substance lies in the range of from 1 to 300 mg, preferably in the range of from 3 to 150 mg, and more preferably in the range of from 6 to 30 mg. And for topical use it lies in the range within 200 mg/g, preferably in the range of from 0.1 to 50 mg/g, and more preferably in the range of from 1 to 15 mg/g.

The daily dosage range for a pangamic acid, given orally to an adult lies in the range of from 2 to 1000 mg, preferably in the range of from 10 to 500 mg, and more preferably in the range of from 20 to 100 mg. And for topical use it lies in the range within 200 mg/g, preferably in the range of from 0.1 to 50 mg/g, and more preferably in the range of from 1 to 15 mg/g.

The daily dosage range for a flavonoid, given orally to an adult lies in the range of from 6 to 1500 mg, preferably in the range of from 30 to 600 mg, and more preferably in the range of from 60 to 300 mg. And for topical use it lies in the range within 200 mg/g, preferably in the range of from 0.1 to 50 mg/g, and more preferably in the range of from 1 to 15 mg/g.

It is possible that the pharmaceutical compositions comprising Epinastine and the antioxidant vitamin are given orally or topically all at once or in divided portions. Topically the formulation is applied directly onto the affected region of skin. Dose adjustment of Epinastine and antioxidant vitamins may reflect age, body weight, and manifesting symptoms.

In a further embodiment the pharmaceutical compositions for the treatment of skin diseases of the present invention comprise antiphlogistics in addition to Epinastine.

Antiphlogistics employed to formulate the present invention are preferably selected form the group of glycyrrhizinic acid (glycyrrhizin) and/or a salt thereof, glycyrrhetinic acid and/or a salt and/or derivative thereof, and/or tranexamic acid and/or a salt thereof. The inventive formulation may comprise one ore more of these antiphlogistics. Theses substances can be used as the neutral compounds or as pharmacologically acceptable salts.

Glycyrrhizinic acid, 20beta-carboxy-11-oxo-30-norolean-12-en-3beta-yl-2-O-beta-D-glucopyranuronosyl-alpha-D-glucopyranosid-uronic acid, is a natural triterpenoid saponine and is a drug that shows antiphlogistic efficacy in the treatment of detoxication, viral, allergic reactions and the like. It has glucocorticoid-like properties. Monomolecular Glycyrrhetinic acid, 3beta-hydroxy-11-oxoolean-12-en-30-oid acid and bimolecular glucuronic acid are other representatives of this chemical family with similar pharmacological properties.

Examples of pharmacologically acceptable salts of glycyrrhizinic acid include dipotassium glycyrrhizinate, potassium glycyrrhizinate, monoammonium glycyrrhizinate, di-ammonium glycyrrhizinate, and the like.

Moreover, examples of the derivatives of glycyrrhetinic acid include glyceryl glycyrrhetinate, stearyl glycyrrhetinate, and the like.

Tranexamic acid, trans-4-(aminomethyl)cyclohexanecarboxylic acid, is a drug showing anti-inflammatory effect, hemostatic action, and antiallergic action by preventing plasmin action.

The compositions comprising Epinastine and the antiphlogistic may also include other pharmacologically active substances such as group B vitamins and vitamin-like active substances such as vitamin B₁, vitamin B₆, vitamin B₁₂, niacin, pantothenic acid, biotin, folic acid, orotic acid, lipoic acid, p-aminobenzoic acid, inositol, carnitine, and choline, antioxidant vitamins and antioxidant vitamin-like substances such as vitamin C, vitamin E, vitamin A, ubiquinone, pangamic acid, and flavonoid, sulfur containing amino acid such as cysteine, methionine, aminoethylsulfonic acid, and glutathione, and vitamin D such as ergocalciferol and cholecalciferol—in addition to Epinastine and the antiphlogistic mentioned above.

Although the amount of the antiphlogistics in the inventive formulation may vary in dependency of the type of the antiphlogistic, the application route etc, the typical amount for oral use given daily to an adult lies in the range from 1 to 2000 mg, and for topical use it lies in the range from 0.1 to 200 mg/g.

Concerning glycyrrhizinic acid and its salt as well as glycyrrhetinic acid the amount for the inventive formulation for oral use given daily to an adult preferably is in the range from 1 to 800 mg, preferably in the range from 2 to 400 mg, more preferably in the range from 15 to 200 mg. For topical use, it lies normally in the range within 200 mg/g, preferably in the range from 0.1 to 50 mg/g, more preferably in the range from 0.2 to 20 mg/g.

Concerning tranexamic acid and its salt the amount for the inventive formulation for oral use given daily to an adult preferably is in the range from 10 to 2000 mg, preferably in the range from 100 to 1000 mg, more preferably in the range from 200 to 750 mg. For topical use, it lies normally in the range within 200 mg/g, preferably in the range. from 1 to 50 mg/g, more preferably in the range from 5 to 20 mg/g.

The pharmaceutical compositions comprising Epinastine and the antiphlogistic may be orally given once or more times, and may be topically applied once or more times directly onto the affected legion of skin. The dose of Epinastine and/or the antiphlogistic may be adjusted in accordance with age, body weight, and manifesting symptoms.

In addition, when the pharmaceutical compositions comprising Epinastine and the antiphlogistic are orally given, the formulations may include a fast release component comprising Epinastine (or Epinastine and part of antiphlogistics) and a slow release component comprising part of or all of the antiphlogistic. When other additional active components are added, they may be added to slow release part and/or the fast release part of the formulation in accordance with the pharmacokinetic characteristic of each active component.

These fast release components and slow release components may be part of one single formulation (unit formulations) or they may be formulated separately in at least two independent formulations. Examples of such unit formulations may include multilayer tablets combining fast release layer(s) and slow release layer(s), granules combining fast release granules and slow release granules or capsules filled with the combination of granules, hard capsules filled with a combination of fast release tablet(s) and slow release tablet(s) both in small size, and dry syrup or suspension syrup using microcapsule or microsphere as slow release components.

All of the pharmaceutical compositions described in the present invention can be used in any oral form such as tablets, granules, fine granules, subtle granules, powders, capsules, caplets, soft capsules, pills, suspensions, emulsions, oral solutions, syrups, dried syrups, chewable forms, forming tablets, effervescent tablets, drops, orally disintegrable tablets, and oral fast-dispersing tablets, and in any topical form such as creams, ointments, gel ointments, suppositories, poultices, tapes, topical solutions, aerosols, lotions, and foams. In addition, preparation formed into microparticles such as microcapsule, nanocapsules, microspheres, nanospheres, liposomes may be also included in the aforementioned compositions.

Moreover, the properties of all of the inventive compositions of the present invention such as stability, release, continuance, disintegration, distinglation, dissolution, concealment of taste, improvement in usage etc. can be regulated by the addition of additives known in the art.

For example, the pharmaceutically active substance can be dispensed in separate granules, multi-layer granules, multi-layer tablets or dry coated tablets, tablets of separated granules, microcapsules, etc. Coating preparations such as sugarcoated tablets, film coating tablets, coating granule, effervescent pharmaceutical preparation can be used as well as chewable preparations, oral fast-dispersing preparations, in the mouth dissolving preparations, matrix preparations, together with comminutions, solid solutions, etc. Sweetening agents, refrigerants, antioxidants or stabilizing agents, agents adjusting a certain pH-value can be added as well as the viscosity, the osmotic pressure or the salt concentration influencing agents. These methods can also be combined.

Optionally, also the following additives can be added: excipients, bases, binders, disintegrators, lubricants, superplasticizers, coating agents, sugar coating agents, plasticizers, antifoaming agents, polish, foaming agents, antistatic agents, desiccant, moisturizing agents, surfactant, solubilizer, buffer agents, resolvents, solubilizing agents, solvents, diluents, stabilizers, emulsifying agents, suspension, suspending agents, dispersing agents, isotonizing agents, aerosol propellant, adsorbents, reducing agents, antioxidant, backing, wetting agents, wet modifier, filler, extender, adhesives, viscous agent, softeners, pH modifiers, antiseptics, preservatives, sweetening agents or preferably bitter taste masking agents like sodium dodecylsulfate (sodium lauryl sulfate), corrigent, refrigerative agents, flavoring agents, perfume, fragrance, coloring matters, and the like. Any of these additives may be used in the regular compositions methods, and do not impose any limitation to such composition methods.

Examples of these additives are explained in the Japanese Pharmaceutical Excipients Directory 2000 (Japan Pharmaceutical Excipients Council edit, Yakuji Nippo. Ltd. issue).

These preparations can be manufactured in the usual manner, i.e. by adding preparation additives to the pharmacologically active substance.

The compositions described in the present invention are explained by examples which follow. However, the present invention of the pharmaceutical compositions is not limited to these examples.

### EXAMPLES

### Example 1

Powder

The following ingredients were homogeneously mixed. The resulted mixed particles were divided into portions of 600 mg to prepare powder compositions.

| | | |
|---|---|---|
| Epinastine hydrochloride | 10 | g |
| L-cysteine | 240 | g |
| Corn starch | 590 | g |
| Lactose | 940 | g |
| Magnesium stearate | 20 | g |

### Example 2

Tablet

The following ingredients were homogeneously mixed. The resulted mixed particles were compressed with a mold to prepare tablets at 120 mg each.

| | | |
|---|---|---|
| Epinastine hydrochloride | 30 | g |
| L-cysteine | 720 | g |
| Lactose | 690 | g |
| Microcrystalline cellulose | 684 | g |
| Light anhydrous silicic acid | 18 | g |
| Talc | 9 | g |
| Magnesium stearate | 9 | g |

### Example 3

Tablet

The following ingredients were homogeneously mixed. The resulted mixed particles were compressed with a mold to prepare tablets at 250 mg each.

| | | |
|---|---|---|
| Epinastine hydrochloride | 20 | g |
| L-methionine | 400 | g |
| Lactose | 510 | g |
| Microcrystalline cellulose | 546 | g |
| Light anhydrous silicic acid | 12 | g |
| Talc | 6 | g |
| Magnesium stearate | 6 | g |

### Example 4

Oral Solution

The following ingredients were dissolved in sterile purified water, added with sodium hydrate to adjust at pH 5, and diluted with sterile purified water to get a total volume of 20 L. The resulted solution was transferred in portions of 50 mL into glass bottles to provide oral solutions.

| | | |
|---|---|---|
| Epinastine hydrochloride | 4 | g |
| Aminoethylsulfonic acid | 400 | g |
| Citric acid | 50 | g |
| Sodium citrate | 10 | g |
| Purified sucrose | 2400 | g |
| Caramel | 60 | g |

| | |
|---|---|
| Sodium hydrate | Adequate amount |
| Antiseptics | Adequate amount |
| Flavor | Trace amount |
| Sterile purified water | Adequate amount |

### Example 5

Syrup

The following ingredients were dissolved in sterile purified water, added with citric acid to adjust at pH 2.5, and then diluted with sterile purified water to prepare syrup at the total volume of 10 L.

| | | |
|---|---|---|
| Epinastine hydrochloride | 20 | g |
| Glutathione | 200 | g |
| Purified sucrose | 4000 | g |
| Sodium chloride | 30 | g |
| Sodium citrate | 20 | g |

| | |
|---|---|
| Citric acid | Adequate amount |
| Antiseptics | Adequate amount |
| Flavor | Trace amount |
| Sterile purified water | Adequate amount |

### Example 6

Sugarcoated Tablet

The following ingredients were processed through a regular method to provide mixed particles, and the particle was compressed to form tablets at 240 mg each.

| | | |
|---|---|---|
| Epinastine hydrochloride | 10 | g |
| L-cysteine | 240 | g |
| Corn starch | 675 | g |
| Lactose | 740 | g |
| Microcrystalline cellulose | 360 | g |
| Hydroxypropylcellulose | 90 | g |
| Light anhydrous silicic acid | 18 | g |
| Talc | 18 | g |
| Magnesium stearate | 9 | g |

Subsequently, the tablets were transferred into a coating pan, and coated using coating solution. The equal volume mixture of ethyl alcohol contained 5% weight/volume of hydroxypropylmethylcellulose and purified water to increase in weight/volume by 10 mg per one tablet. Next, 2% weight/volume of talc, 2% weight/volume of titanium oxide, 3% weight/volume of calcium carbonate, 1% weight/volume of powdered acacia, and aqueous solution containing 60% weight/volume of purified sucrose were used to coat tablets to give increase in weight/volume by 150 mg per one tablet. Finally, aqueous solution containing 60% weight/volume purified sucrose was used to coat tablets to give an increase in weight/volume by 30 mg per one tablet. Thus sugarcoated tablets were prepared.

### Example 7

Granules

The following ingredients were prepared as granules through a regular method to prepare mixed particles, and packed to give an amount of 1000 mg per one pack for granules.

| | | |
|---|---|---|
| Epinastine hydrochloride | 10 | g |
| DL-methionine | 1000 | g |
| Calcium carboxymethylcellulose | 240 | g |
| Mannitol | 1100 | g |
| Corn starch | 508 | g |
| Tartaric acid | 100 | g |
| Aspartame | 20 | g |
| Acesulfame potassium | 20 | g |
| Fragrant materials | 2 | g |

### Example 8

Cream

The following ingredients were processed through a regular method to form a cream of a total weight of 1 kg, added with sodium citrate to adjust at pH 5.

| | | |
|---|---|---|
| Epinastine hydrochloride | 10.0 | g |
| L-cysteine | 1.0 | g |
| Medium chain fatty acid triglyceride | 200.0 | g |
| Propylene glycol | 150.0 | g |
| Glyceryl monostearate | 80.0 | g |
| Polyoxyethylene cetyl ether | 40.0 | g |
| Diisopropyl adipate | 50.0 | g |
| Citric acid | 0.1 | g |

| | |
|---|---|
| Sodium citrate | Adequate amount |
| Antiseptics | Adequate amount |
| Purified water | Adequate amount |

Any of the following examples 9 to 16 may comprise a sweetener or preferably a bitter taste masking agent, like for example sodium dodecylsulfate (sodium lauryl sulfate) in an amount of less than 300 mg for a daily dosage.

### Example 9

Powder

The following ingredients were homogeneously mixed. The resulted mixed particles were divided into portions of 800 mg to prepare powder compositions.

| | | |
|---|---|---|
| Epinastine hydrochloride | 20.0 | g |
| Riboflavin | 24.0 | g |
| Pyridoxine hydrochloride | 100.0 | g |
| Calcium pantothenate | 60.0 | g |
| L-cysteine | 320.0 | g |
| Biotin | 0.1 | g |
| Orotic acid | 400.0 | g |
| Thioctic acid amide | 20.0 | g |
| p-Aminobenzoic acid | 600.0 | g |
| Corn starch | 1167.9 | g |
| Lactose | 2040.0 | g |
| Magnesium stearate | 48.0 | g |

### Example 10

Tablet

The following ingredients were homogeneously mixed. The resulted mixed particles were compressed with a mold to prepare tablets at 150 mg each.

| | | |
|---|---|---|
| Epinastine hydrochloride | 30 | g |
| Thiamin nitrate | 45 | g |
| Riboflavin butyrate | 36 | g |
| Pyridoxine hydrochloride | 135 | g |
| Nicotinamide | 450 | g |
| Calcium pantothenate | 90 | g |
| Lactose | 933 | g |
| Microcrystalline cellulose | 945 | g |
| Light anhydrous silicic acid | 18 | g |
| Talc | 9 | g |

### Example 11

Tablet

The following ingredients were homogeneously mixed. The resulted mixed particles were compressed with a mold to prepare tablets at 250 mg each.

| | | |
|---|---|---|
| Epinastine hydrochloride | 20 | g |
| Pyridoxal phosphate | 24 | g |
| Riboflavin butyrate | 24 | g |
| Inositol | 36 | g |
| Aminoethyl sulfonic acid | 72 | g |
| Panthenol | 120 | g |
| Carnitine chloride | 100 | g |
| Biotin | 1 | g |
| Folic acid | 20 | g |
| Lactose | 513 | g |
| Microcrystalline cellulose | 546 | g |
| Light anhydrous silicic acid | 12 | g |
| Talc | 6 | g |
| Magnesium stearate | 6 | g |

### Example 12

Oral Solution

The following ingredients were dissolved in sterile purified water, added with sodium hydrate to adjust at pH 5, and diluted with sterile purified water to get a total volume of 20 L. The resulted solution was transferred in portions of 50 mL into glass bottles to provide oral solutions.

| | | |
|---|---|---|
| Epinastine hydrochloride | 4 | g |
| Aminoethylsulfonic acid | 80 | g |
| Inositol | 20 | g |
| Thiamin nitrate | 4 | g |
| Riboflavin sodium phosphate | 4 | g |
| Pyridoxine hydrochloride | 4 | g |
| Carnitine chloride | 40 | g |
| Nicotinamide | 10 | g |
| Calcium pantothenate | 8 | g |
| Orotic acid choline | 40 | g |
| Cyanocobalamin | 0.004 | g |
| Thioctic acid | 2 | g |
| Citric acid | 50 | g |
| Sodium citrate | 10 | g |
| Purified sucrose | 2400 | g |
| Caramel* | 60 | g |

| | |
|---|---|
| Sodium hydrate | Adequate amount |
| Antiseptics | Adequate amount |
| Flavor | Trace amount |
| Sterile purified water | Adequate amount |

* instead of Caramel sodium dodecylsulfate in an amount of up to 300 mg per day may be used.

### Example 13

Syrup

The following ingredients were dissolved in sterile purified water, added with citric acid to adjust at pH 2.5, and then diluted with sterile purified water to prepare syrup at the total volume of 10 L.

| | | |
|---|---|---|
| Epinastine hydrochloride | 20 | g |
| Pyridoxine hydrochloride | 20 | g |
| Riboflavin sodium phosphate | 40 | g |
| Panthenol | 60 | g |
| Purified sucrose | 4000 | g |
| Sodium chloride | 30 | g |
| Sodium citrate | 20 | g |

| | |
|---|---|
| Citric acid | Adequate amount |
| Antiseptics | Adequate amount |
| Flavor | Trace amount |
| Sterile purified water | Adequate amount |

### Example 14

Sugarcoated Tablet

The following ingredients were processed through a regular method to provide mixed particles, and the particle was compressed to form tablets at 250 mg each.

| | | |
|---|---|---|
| Epinastine hydrochloride | 10 | g |
| Calcium pantothenate | 15 | g |
| Ascorbic acid | 200 | g |
| L-cysteine | 160 | g |
| Corn starch | 630 | g |
| Lactose | 740 | g |
| Microcrystalline cellulose | 360 | g |
| Hydroxypropylcellulose | 90 | g |
| Light anhydrous silicic acid | 18 | g |
| Talc | 18 | g |
| Magnesium stearate | 9 | g |

Subsequently, the tablets were transferred into a coating pan, and coated using coating solution. The equal volume mixture of ethyl alcohol contained 5% weight/volume of hydroxypropylmethylcellulose and purified water to increase in weight/volume by 10 mg per one tablet. Next, 2% weight/volume of talc, 2% weight/volume of titanium oxide, 3% weight/volume of calcium carbonate, 1% weight/volume of powdered acacia, and aqueous solution containing 60% weight/volume of purified sucrose were used to coat tablets to give increase in weight/volume by 150 mg per one tablet. Finally, aqueous solution containing 60% weight/volume purified sucrose was used to coat tablets to give an increase in weight/volume by 30 mg per one tablet. Thus sugarcoated tablets were prepared.

### Example 15

Granules

The following ingredients were prepared as granules through a regular method to prepare mixed particles, and packed to give an amount of 1000 mg per one pack for granules.

| | | |
|---|---|---|
| Epinastine hydrochloride | 10 | g |
| Thiamin nitrate | 5 | g |
| Riboflavin | 5 | g |
| Pyridoxine hydrochloride | 10 | g |
| Nicotinamide | 10 | g |
| DL-methionine | 1000 | g |
| Calcium carboxymethylcellulose | 240 | g |
| Mannitol | 1100 | g |
| Corn starch | 478 | g |
| Tartaric acid | 100 | g |
| Aspartame* | 20 | g |
| Acesulfame potassium | 20 | g |
| Fragrant materials | 2 | g |

*instead of aspartame sodium dodecylsulfate in an amount of up to 300 mg per day may be used.

### Example 16

Cream

The following ingredients were processed through a regular method to form a cream of a total weight of 1 kg, added with sodium citrate to adjust at pH 5.

| | | |
|---|---|---|
| Epinastine hydrochloride | 10.0 | g |
| Pyridoxine hydrochloride | 1.0 | g |
| Tocopherol acetate | 10.0 | g |
| Medium chain fatty acid triglyceride | 200.0 | g |
| Propylene glycol | 150.0 | g |
| Glyceryl monostearate | 80.0 | g |
| Polyoxyethylene cetyl ether | 40.0 | g |
| Diisopropyl adipate | 50.0 | g |
| Citric acid | 0.1 | g |

| | |
|---|---|
| Sodium citrate | Adequate amount |
| Antiseptics | Adequate amount |
| Purified water | Adequate amount |

### Example 17

Powder

The following ingredients were uniformly mixed. The resulted mixed particles were divided into 600 mg per one pack to prepare powder compositions.

| | | |
|---|---|---|
| Epinastine hydrochloride | 10 | g |
| Calcium ascorbate | 180 | g |
| L-cysteine | 160 | g |
| Corn starch | 530 | g |
| Lactose | 900 | g |
| Magnesium stearate | 20 | g |

### Example 18

Granules

The following ingredients were prepared into granules through a regular method to prepare mixed particles, and packed to give amount of 1000 mg per one pack for granules.

| | | |
|---|---|---|
| Epinastine hydrochloride | 10 | g |
| Ascorbic acid | 250 | g |
| Calcium ascorbate | 250 | g |
| Riken Dry A-S200PT (Vitamin A 200,000 I.U./g) | 0.01 | g |
| dl-α-tocopherol calcium succinate | 100 | g |
| Ubiquinone | 30 | g |
| Pangamic acid | 50 | g |
| Flavonoid | 100 | g |
| Calcium carboxymethylcellulose | 240 | g |
| Mannitol | 1300 | g |
| Corn starch | 527.99 | g |
| Tartaric acid | 100 | g |
| Aspartame | 20 | g |
| Acesulfame potassium | 20 | g |
| Fragrant materials | 2 | g |

### Example 19

Tablet

The following ingredients were uniformly mixed. The resulted mixed particles were compressed with a mold to prepare tablets at 250 mg each.

| | | |
|---|---|---|
| Epinastine hydrochloride | 30 | g |
| dl-α-tocopherol calcium succinate | 250 | g |
| Ubiquinone | 75 | g |
| Lactose | 310 | g |
| Microcrystalline cellulose | 575 | g |
| Light anhydrous silicic acid | 5 | g |
| Talc | 5 | g |
| Magnesium stearate | 5 | g |

### Example 20

Tablet

The following ingredients were uniformly mixed. The resulted mixed particles were compressed with a mold to prepare tablets at 250 mg each.

| | | |
|---|---|---|
| Epinastine hydrochloride | 20 | g |
| Ascorbic acid | 200 | g |
| dl-α-tocopherol calcium succinate | 200 | g |
| Riken Dry A-S200PT (Vitamin A 200,000 I.U./g) | 0.02 | g |
| Lactose | 455.98 | g |
| Microcrystalline cellulose | 600 | g |
| Light anhydrous silicic acid | 12 | g |
| Talc | 6 | g |
| Magnesium stearate | 6 | g |

### Example 21

Oral Solution

The following ingredients were dissolved into a portion of sterile purified water, added with sodium hydrate to adjust at pH 5, and diluted with sterile purified water to make total volume of 20 L. The resulted solution was transferred by 50 mL into glass bottles to provide oral solutions.

| | | |
|---|---|---|
| Epinastine hydrochloride | 4 | g |
| Ascorbic acid | 40 | g |
| Aminoethylsulfonic acid | 400 | g |
| Citric acid | 50 | g |
| Sodium citrate | 10 | g |
| Purified sucrose | 2400 | g |
| Caramel | 60 | g |

| | |
|---|---|
| Sodium hydrate | Adequate amount |
| Antiseptics | Adequate amount |
| Flavor | Trace amount |
| Sterile purified water | Adequate amount |

### Example 22

Cream

The following ingredients were processed through a regular method to form cream at the total weight of 1 kg, added with sodium citrate to adjust at pH 5.

| | | |
|---|---|---|
| Epinastine hydrochloride | 10.0 | g |
| dl-a-tocopherol acetate | 5.0 | g |
| Vitamin A oil: vitamin A 100000 I.U./g | 2.0 | g |
| Medium chain fatty acid triglyceride | 200.0 | g |
| Propylene glycol | 150.0 | g |
| Glyceryl monostearate | 80.0 | g |
| Polyoxyethylene cetyl ether | 40.0 | g |
| Diisopropyl adipate | 50.0 | g |
| Citric acid | 0.1 | g |

| | |
|---|---|
| Sodium citrate | Adequate amount |
| Antiseptics | Adequate amount |
| Purified water | Adequate amount |

### Example 23

Tablet

The following ingredients were homogeneously mixed. The resulting mixed particles were compressed with a mold to prepare tablets of 250 mg each.

| | | |
|---|---|---|
| Epinastine hydrochloride | 20 | g |
| Potassium glycyrrhizinate | 360 | g |
| Pyridoxine hydrochloride | 45 | g |
| Nicotinamide | 450 | g |
| Calcium pantothenate | 60 | g |
| Lactose | 481 | g |
| Microcrystalline cellulose | 506 | g |
| Light anhydrous silicic acid | 14 | g |
| Magnesium stearate | 10 | g |
| Talc | 4 | g |

### Example 24

Powder

The following ingredients were homogeneously mixed. The resulted mixed particles were divided into portions of 600 mg to prepare powder compositions.

| | | |
|---|---|---|
| Epinastine hydrochloride | 5 | g |
| Tranexamic acid | 375 | g |
| Corn starch | 238 | g |
| Lactose | 270 | g |
| Magnesium stearate | 12 | g |

### Example 25

Two Layer Tablet Comprising an A Layer and a B Layer

The following ingredients of A layer and B layer were processed according to a standard method to provide mixed particles, respectively, and the particles were compressed to form

| A layer | | |
|---|---|---|
| Epinastine hydrochloride | 60 | g |
| Lactose | 258 | g |
| Microcrystalline cellulose | 270 | g |
| Light anhydrous silicic acid | 6 | g |
| Talc | 3 | g |
| Magnesium stearate | 3 | g |
| B layer | | |
| Monoammonium glycyrrhizinate | 360 | g |
| Pyridoxine hydrochloride | 72 | g |
| Lactose | 330 | g |
| Hydrogenated oil | 84 | g |
| Hydroxypropylmethylcellulose 2208 | 45 | g |
| Magnesium stearate | 9 | g |

### Example 26

Oral Solution

The following ingredients were dissolved in sterile purified water, added with sodium hydrate to adjust at pH 5, and diluted with sterile purified water to get a total volume of 20 L. The resulted solution was transferred in portions of 50 mL into glass bottles to provide oral solutions.

| | | |
|---|---|---|
| Epinastine hydrochloride | 4 | g |
| Glycyrrhizinic acid | 40 | g |
| Aminoethylsulfonic acid | 80 | g |
| Inositol | 20 | g |
| Thiamin nitrate | 4 | g |
| Riboflavin sodium phosphate | 4 | g |
| Pyridoxine hydrochloride | 4 | g |
| Carnitine chloride | 40 | g |
| Nicotinamide | 10 | g |
| Calcium pantothenate | 8 | g |
| Citric acid | 50 | g |
| Sodium citrate | 10 | g |
| Purified sucrose | 2400 | g |
| Caramel | 60 | g |

| | |
|---|---|
| Sodium hydrate | Adequate amount |
| Antiseptics | Adequate amount |
| Flavor | Trace amount |
| Sterile purified water | Adequate amount |

### Example 27

Syrup

The following ingredients were dissolved in sterile purified water, added with citric acid to adjust a pH of 2.5. Then they were diluted with sterile purified water to prepare syrup at the total volume of 10 L.

| | | |
|---|---|---|
| Epinastine hydrochloride | 20 | g |
| Dipotassium glycyrrhizinate | 220 | g |
| Pyridoxine hydrochloride | 20 | g |
| Riboflavin sodium phosphate | 40 | g |
| Panthenol | 60 | g |
| Purified sucrose | 4000 | g |
| Sodium chloride | 30 | g |
| Sodium citrate | 20 | g |

| | |
|---|---|
| Citric acid | Adequate amount |
| Antiseptics | Adequate amount |
| Flavor | Trace amount |
| Sterile purified water | Adequate amount |

### Example 28

Sugarcoated Tablet

The following ingredients were processed according to a standard method to provide mixed particles, and the particle was compressed to form tablets at 240 mg each.

| | | |
|---|---|---|
| Epinastine hydrochloride | 10 | g |
| Dipotassium glycyrrhizinate | 200 | g |
| L-cysteine | 120 | g |
| Ascorbic acid | 100 | g |
| Pyridoxine hydrochloride | 50 | g |
| Calcium pantothenate | 30 | g |
| Riboflavin butyrate | 12 | g |
| Lactose | 640 | g |
| Corn starch | 406 | g |
| Microcrystalline cellulose | 306 | g |
| Low substituted hydroxypropylcellulose | 130 | g |
| Hydroxypropylcellulose | 90 | g |
| Light anhydrous silicic acid | 45 | g |
| Talc | 12 | g |
| Magnesium stearate | 9 | g |

Subsequently, the tablets were transferred into a coating pan, and coated using coating solution. The equal volume mixture of ethyl alcohol contained 5% weight/volume of hydroxypropylmethylcellulose and purified water to increase in weight/volume by 10 mg per one tablet. Next, 2% weight/volume of talc, 2% weight/volume of titanium oxide, 3% weight/volume of calcium carbonate, 1% weight/volume of powdered acacia, and aqueous solution containing 60% weight/volume of purified sucrose were used to coat tablets to give increase in weight/volume by 100 mg per one tablet. Finally, aqueous solution containing 60% weight/volume purified sucrose was used to coat tablets to give an increase in weight/volume by 100 mg per one tablet. Thus sugarcoated tablets were prepared.

### Example 29

Granules

The following ingredients were prepared as granules according to a standard method to prepare mixed particles, and packed to give an amount of 1000 mg per one pack for granules.

| | | |
|---|---|---|
| Epinastine hydrochloride | 10 | g |
| Glycyrrhizinic acid | 90 | g |
| Thiamin nitrate | 5 | g |
| Riboflavin | 5 | g |
| Pyridoxine hydrochloride | 6 | g |
| Nicotinamide | 30 | g |
| Orotic acid | 90 | g |
| Hesperidin | 120 | g |
| DL-methionine | 100 | g |
| Calcium carboxymethylcellulose | 240 | g |
| Mannitol | 1500 | g |
| Corn starch | 662 | g |
| Tartaric acid | 100 | g |
| Aspartame | 20 | g |
| Acesulfame potassium | 20 | g |
| Fragrant materials | 2 | g |

### Example 30

Cream

The following ingredients were processed according to a standard method to form a cream of a total weight of 1 kg, added with sodium citrate to adjust at pH 5.

| | | |
|---|---|---|
| Epinastine hydrochloride | 10.0 | g |
| Glycyrrhetinic acid | 10.0 | g |
| Pyridoxine hydrochloride | 1.0 | g |
| Medium chain fatty acid triglyceride | 200.0 | g |
| Propylene glycol | 150.0 | g |
| Glyceryl monostearate | 80.0 | g |
| Polyoxyethylene cetyl ether | 40.0 | g |
| Diisopropyl adipate | 50.0 | g |
| Citric acid | 0.1 | g |

| | |
|---|---|
| Sodium citrate | Adequate amount |
| Antiseptics | Adequate amount |
| Purified water | Adequate amount |

### Example 31

Ointment

The following ingredients were processed according to a standard method to form an ointment of a total weight of 1 kg.

| | | |
|---|---|---|
| Epinastine hydrochloride | 10 | g |
| Glycyrrhetinic acid | 10 | g |
| Crotamiton | 100 | g |
| Lidocaine | 20 | g |
| Chlorhexidine hydrochloride | 2 | g |
| Paraffin | 40 | g |
| Cetanol | 30 | g |
| White beeswax | 30 | g |

| | |
|---|---|
| White petrolatum | Adequate amount |

1. A pharmaceutical composition comprising Epinastine or a pharmaceutically acceptable salt thereof as a pharmacologically active compound and at least one compound selected from the group consisting of a) one or more sulfur containing amino acids or peptides, b) one or more vitamins of the vitamin B group, c) one or more vitamins having antioxidant properties and d) one or more antiphlogistic compounds or pharmaceutically acceptable salts, derivatives or mixtures thereof.

2. The pharmaceutical composition according to claim 1, wherein the Epinastine salt is selected from the group consisting of hydrochloride, hydrobromide, oxalate, nitrate, sulfonate, fumarate, maleate, sulfate and phosphate.

3. The pharmaceutical composition according to claim 2, wherein the Epinastine salt is hydrochloride.

4. The pharmaceutical composition according to claim 1, wherein one or more sulfur containing amino acids or peptides is selected from the group consisting of cystine, methionine, aminoethylsulfonic acid, glutathione, cystine, homocysteine, homocystine, cysteine sulfinic acid, and lanthionine or pharmaceutically acceptable salts, derivatives or mixtures thereof.

5. The pharmaceutical composition according to claim 4, wherein one or more sulfur containing amino acids or peptides is selected from the group consisting of cystine, methionine, taurine and glutathione or pharmaceutically acceptable salts, derivatives or mixtures thereof.

6. The pharmaceutical composition according to claim 1, wherein one or more vitamins of the vitamin B group is selected from the group consisting of vitamin B₁, vitamin B₂, vitamin B₆, vitaminB₁₂, niacin, pantothenic acid, biotin, folic acid, orotic acid, thioctic acid, p-aminobenzoic acid, inositol, carnitine and choline or pharmaceutically acceptable salts, derivatives or mixtures thereof.

7. The pharmaceutical composition according to claim 1, wherein one or more vitamins having antioxidant properties is selected from the group consisting of vitamin C, vitamin E, vitamin A, and antioxidant vitamin-like substances or pharmaceutically acceptable salts, derivatives or mixtures thereof.

8. The pharmaceutical composition according to claim 7, wherein one or more vitamins having antioxidant properties is selected from the group consisting of vitamin C, vitamin E and vitamin A or pharmaceutically acceptable salts, derivatives or mixtures thereof.

9. The pharmaceutical composition according to claim 1, wherein one or more antiphogilistic compounds is selected from the group consisting of glycyrrhizinic acid, glycyrrhetinic acid and tranexamic acid and pharmaceutically acceptable salts, derivatives or mixtures thereof.

10. A method of treating skin disease associated with allergic reactions comprising administering to a patient in need thereof an effective amount of the composition according to claim 1.

11. The method according to claim 10, wherein the composition is administered orally.

12. The method according to claim 10, wherein the composition is administered topically.

13. The pharmaceutical composition according to claim 1, further comprising additives

14. A pharmaceutical composition comprising Epinastine or a pharmaceutically acceptable salt thereof as a pharmacologically active compound and one or more sulfur containing amino acids or peptides or pharmaceutically acceptable salts, derivatives or mixtures thereof.

15. The pharmaceutical composition according to claim 14, wherein one or more sulfur containing amino acids or peptides is selected from the group consisting of cystine, methionine, aminoethylsulfonic acid, glutathione, cystine, homocysteine, homocystine, cysteine sulfinic acid, and lanthionine or pharmaceutically acceptable salts, derivatives or mixtures thereof.

16. The pharmaceutical composition according to claim 14, wherein one or more sulfur containing amino acids or peptides is selected from the group consisting of cystine, methionine, taurine and glutathione or pharmaceutically acceptable salts, derivatives or mixtures thereof.

17. A method of treating skin disease associated with allergic reactions comprising administering to a patient in need thereof an effective amount of the composition according to claim 14.

18. The method according to claim 17, wherein the composition is administered orally.

19. The method according to claim 18, wherein the amount of Epinastine is between 2 and 20 mg.

20. The method according to claim 18, wherein the amount of one or more sulfur containing amino acids or peptides is between 5 and 10000 mg.

21. The method according to claim 17, wherein the composition is administered topically.

22. The method according to claim 21, wherein the amount of Epinastine is between 1 and 50 mg.

23. The method according to claim 21, wherein the amount of one or more sulfur containing amino acids or peptides is between 0.01 and 200 mg.

24. The pharmaceutical composition according to 14, further comprising additives.

25. A pharmaceutical composition comprising Epinastine or a pharmaceutically acceptable salt thereof as a pharmacologically active compound and one or more vitamins of the vitamin B group or pharmaceutically acceptable salts, derivatives or mixtures thereof.

26. The pharmaceutical composition according to claim 25, wherein one or more vitamins of the vitamin B group is selected from the group consisting of vitamin B₁, vitamin B₂, vitamin B₆, vitaminB₁₂, niacin, pantothenic acid, biotin, folic acid, orotic acid, thioctic acid, p-aminobenzoic acid, inositol, camitine and choline or pharmaceutically accepatble salts, derivatives or mixtures thereof.

27. A method of treating skin disease associated with allergic reactions comprising administering to a patient in need thereof an effective amount of the composition according to claim 25.

28. The method according to claim 27, wherein the composition is administered orally.

29. The method according to claim 28, wherein the amount of Epinastine is between 2 and 20 mg.

30. The method according to claim 28, wherein the amount of the one or more vitamins from the vitamin B group is between 0.0001 and 1500 mg.

31. The method according to claim 27, wherein the composition is administered topically.

32. The method according to claim 31, wherein the amount of Epinastine is between 1 and 50 mg.

33. The method according to claim 31, wherein the amount of one or more vitamins from the vitamin B group is between 0.01 and 200 mg.

34. The pharmaceutical composition according to claim 25, further comprising additives.

35. A pharmaceutical composition comprising Epinastine or a pharmaceutically acceptable salt thereof as a pharmacologically active compound and one or more vitamins having antioxidant properties or pharmaceutically acceptable salts, derivatives or mixtures thereof.

36. The pharmaceutical composition according to claim 35, wherein one or more vitamins having antioxidant properties is selected from the group consisting of vitamin C, vitamin E, vitamin A, and antioxidant vitamin-like substances or pharmaceutically acceptable salts, derivatives or mixtures thereof.

37. The pharmaceutical composition according to claim 36, wherein one or more vitamins having antioxidant properties is selected from the group consisting of vitamin C, vitamin E and vitamin A or pharmaceutically acceptable salts, derivatives or mixtures thereof.

38. A method of treating skin disease associated with allergic reactions comprising administering to a patient in need thereof an effective amount of the composition according to claim 35.

39. The method according to claim 38, wherein the composition is administered orally.

40. The method according to claim 39, wherein the amount of Epinastine is between 2 and 20 mg.

41. The method according to claim 39, wherein the amount of one or more vitamins having antioxidant properties is between 0.01 and 3000 mg.

42. The method according to claim 38, wherein the composition is administered topically.

43. The method according to claim 42, wherein the amount of Epinastine is between 1 and 50 mg.

44. The method according to claim 42, wherein the amount of one or more vitamins having antioxidant properties is between 0.1 and 200 mg.

45. The pharmaceutical composition according to claim 35, further comprising additives.

46. A pharmaceutical composition comprising Epinastine or a pharmaceutically acceptable salt thereof as a pharmacologically active compound and one or more antiphlogistic compounds or pharmaceutically acceptable salts, derivatives or mixtures thereof.

47. The pharmaceutical composition according to claim 46, wherein one or more antiphlogistic compounds is selected from the group consisting of glycyrrhizinic acid, glycyrrhetinic acid and tranexamic acid or pharmaceutically acceptable salts, derivatives or mixtures thereof.

48. A method of treating skin disease associated with allergic reactions comprising administering to a patient in need thereof an effective amount of the composition according to claim 46.

49. The method according to claim 48, wherein the composition is administered orally.

50. The method according to claim 49, wherein the amount of Epinastine is between 2 and 20 mg.

51. The method according to claim 49, wherein the amount of one or more antiphlogistic compounds is between 1 and 2000 mg.

52. The method according to claim 48, wherein the composition is administered topically.

53. The method according to claim 52, wherein the amount of Epinastine is between 1 and 50 mg.

54. The method according to claim 52, characterized in that the amount of one or more antiphlogistic compounds is between 0.1 and 200 mg.

55. The pharmaceutical composition according to claim 46, further comprising additives.
